(19) United States
(12) Patent Application Publication
(10) Pub. No.: US 2015/0032203 A1
(43) Pub. Date: Jan. 29, 2015

(54) FLEXIBLE VASCULAR PROSTHESIS, AND METHOD FOR ITS PRODUCTION

(71) Applicant: Aesculap AG, Tuttlingen (DE)

(72) Inventors: Jan-Philip Zappe, Lohfelden (DE); Dietmar Probst, Melsungen (DE)

(21) Appl. No.: 14/374,264

(22) PCT Filed: Jan. 24, 2013
(86) PCT No.: PCT/EP2013/051364

§ 371 Date: Jul. 24, 2014

(30) Foreign Application Priority Data
Jan. 25, 2012 (DE) 10 2012 201 094.7

(51) Int. Cl. A61F 2/06; A61L 17/14; A61F 2/82

(52) U.S. Cl. CPC A61F 2/06; A61F 2/82; A61L 17/145; A61L 2300/606; A61L 2400/18; A61L 2420/02
USPC 623/1.45; 427/2.25

(57) ABSTRACT

A vascular prosthesis with an impregnation including at least one sealing material in the prosthesis wall and/or with a coating including at least one sealing material on the outer surface of the prosthesis, wherein the prosthesis, when subjected to a force of 1 N acting perpendicularly with respect to the outer surface of the prosthesis, is deformed such that the external diameter of the prosthesis decreases, in the direction of the acting force, by 60 to 100% in relation to the original external diameter.

### TECHNICAL FIELD

This disclosure relates to a flexible, in particular soft, vascular prosthesis, and to a method for its production.

### BACKGROUND

Vascular prostheses generally have a porous main structure to prevent incorporation of body cells and tissue. This, on the one hand, permits secondary anchoring of the prosthesis after its implantation. On the other hand, the growth of body cells and tissue into the prostheses allows a substantial approximation to the original anatomical circumstances.

However, porosity of the prosthesis structure is generally associated with the risk of leaks occurring, which can in turn be the cause of undesired and in particular life-threatening seepage of blood.

To avoid leaks, vascular prostheses can undergo so-called “pre-clotting.” Such vascular prostheses are soaked with the patient's blood before the operation. A vascular prosthesis suitable for this purpose is known from WO 02/094135 A1, for example.

However, since pre-clotting constitutes a time-consuming preliminary treatment of the vascular prosthesis, it is increasingly common for vascular prostheses to be impregnated or coated with resorbable materials. Vascular prostheses of that kind permit a broader range of application and can in particular also be used for emergency operations.

For example, a thin and lacquer-like coating with an antithrombogenic action is known, for biomaterials such as stents, from EP 0 652 017 A1.

A vascular prosthesis coated with a synthetic resorbable polymer is the subject matter of DE 10 2006 053 752 A1.

DE 10 2009 037 134 A1 discloses a vascular prosthesis with a polyurethane coating. U.S. Pat. No. 6,733,768 B2 describes prosthesis-coating compositions that contain polymers and active substances.

A vascular prosthesis impregnated by gelatin, in which substantially the entire prosthesis wall is impregnated, is known from DE 101 49 392 A1.

Generally, to generate a sealing coating, the prostheses undergo an immersion process. The coatings resulting from the latter are often film-shaped and can lead to increased stiffness and hardness of the prosthesis.

In contrast, if the vascular prostheses are coated by a spraying process, a large number of spray cycles is generally needed to form a sealing coating, which can likewise result in stiffer overall prosthesis constructions.

Stiffer vascular prostheses, however, make handling difficult for the surgeon since they are more difficult to adapt during the implantation procedure and to sew onto natural tissues.

Against this background, it could be helpful to provide a method by which a vascular prosthesis is produced and which avoids the known shortcomings.

It could also be helpful to provide a vascular prosthesis with improved flexibility and softness, improved ease of handling by the surgeon, and at the same time by a reduced risk of postoperative complications.

### SUMMARY

We provide a method of producing a vascular prosthesis including spraying a vascular prosthesis with an inner surface and outer surface and a wall with a liquid containing at least one sealing material, wherein the liquid is sprayed onto the outer surface of the prosthesis at a pressure of 0.01 to 1.5 bar to generate a sealing impregnation in the prosthesis wall, and/or is sprayed onto the outer surface of the prosthesis at a pressure of 5.0 to 50 bar to generate a sealing coating on the outer surface of the prosthesis.

We also provide a vascular prosthesis with an impregnation including at least one sealing material in the prosthesis wall and/or with a coating including at least one sealing material on the outer surface of the prosthesis, wherein the prosthesis, when subjected to a force of 1 N acting perpendicularly with respect to the outer surface of the prosthesis, is deformed such that the external diameter of the prosthesis decreases, in the direction of the acting force, by 60 to 100% in relation to the original external diameter.

### BRIEF DESCRIPTION OF THE DRAWINGS

FIG. 1 shows a cross section of the wall of a vascular prosthesis with an impregnation in the prosthesis wall.

FIG. 2 shows a cross section of the wall of a vascular prosthesis with a coating on the outer surface of the prosthesis.

FIG. 3 shows a cross section of the wall of a vascular prosthesis with an impregnation in the prosthesis wall and a coating on the outer surface of the prosthesis.

FIG. 4 shows a graph indicating the results of softness measurements carried out on vascular prostheses.

### DETAILED DESCRIPTION

We provide a method of producing a vascular prosthesis in which a vascular prosthesis with an inner surface and outer surface and a wall is sprayed with a liquid containing at least one sealing material.

To generate a sealing impregnation in the prosthesis wall, the liquid is sprayed onto the outer surface of the prosthesis at a pressure of 0.01 to 1.5 bar.

Alternatively or in combination, the liquid is sprayed onto the outer surface of the prosthesis at a pressure of 5.0 to 50 bar to generate a sealing coating on the outer surface of the prosthesis.

We discovered the following surprising results:

If the outer surface of a vascular prosthesis is sprayed with a liquid, containing at least one sealing material, at a pressure of 0.01 to 1.5 bar, a sealing impregnation forms in the prosthesis wall and acts in particular as a kind of substrate or adhesive layer (grounding or priming coat) with respect to a coating optionally present on the outer surface of the prosthesis.

If, alternatively or in addition, the outer surface of a vascular prosthesis is sprayed with a liquid containing at least one sealing material at a pressure of 5.0 to 50 bar, a sealing coating forms on the outer surface of the prosthesis and in particular leads to prosthesis flexibility, preferably prosthesis softness, that is much greater than in conventionally coated prostheses with a comparable coating fraction. The flexibility of prostheses coated by our method is (in some cases) even comparable to the flexibility of uncoated prostheses.

The greater flexibility, in particular softness, of the prostheses improves their handling by the surgeon. For example, our vascular prostheses permit easier adaptation to natural blood vessels, which is advantageous especially in bypass operations. Fastening of the prostheses to natural blood vessels is also improved by the greater flexibility.

Moreover, it is possible to do without after-treatment, for example, restoration of pleats.

Finally, the pressure conditions provided permit a more efficient spraying process, which is reflected particularly in a reduced number of spray cycles.

The expression “at least one sealing material” can signify a sealing material in the sense of a single type of sealing material or a plurality or mixture of different sealing materials.

The expression “sealing impregnation” or “sealing coating” signifies that the impregnation or coating is leaktight with respect to body fluids, preferably blood.

The expression “impregnation” is to be understood as at least one layer which contains the at least one sealing material and formed in the wall of the prosthesis preferably to a depth of at least 1 to 100%, in particular 50 to 100%, preferably 80 to 100%, relative to the wall thickness of the prosthesis. If appropriate, the at least one layer is in addition also formed on the outer surface of the prosthesis. If the impregnation is formed in the prosthesis wall and on the outer surface of the prosthesis, the layer thickness of the impregnation in the prosthesis wall is preferably greater than on the outer surface of the prosthesis. Preferably, the impregnation is formed only, or substantially only, in the prosthesis wall.

The expression “coating” is to be understood as at least one layer containing the at least one sealing material and formed in the wall of the prosthesis preferably to a depth of at most 15%, in particular at most 8 to 12%, preferably at most 9%, relative to the wall thickness of the prosthesis, and is for the rest formed on the outer surface of the prosthesis. If the coating is formed on the outer surface of the prosthesis and in the prosthesis wall, the thickness of the coating on the outer surface of the prosthesis is preferably greater than in the prosthesis wall. Preferably, the coating is formed only, or substantially only, on the outer surface of the prosthesis.

The term “at least one layer” mentioned in the two previous paragraphs can signify one layer or a multiplicity of layers, i.e. at least two or more layers.

Preferably the liquid that generates the impregnation is sprayed onto the outer surface of the vascular prosthesis at a pressure of 0.02 to 1.5 bar, in particular 0.05 to 0.8 bar, preferably 0.1 to 0.6 bar.

Alternatively or additionally, the liquid that generates the coating is sprayed onto the outer surface of the vascular prosthesis at a pressure of 5 to 20 bar, in particular 5 to 10 bar, preferably 6 to 9 bar.

The liquid may be applied to the outer surface of the vascular prosthesis by a spraying device controlled by compressed air, preferably a spray gun.

The outer surface of the vascular prosthesis is preferably sprayed with the liquid at a distance of 1 to 500 mm, in particular 1 to 100 mm, preferably 2 to 70 mm, from the spraying device.

1 to 100 spray cycles can be performed to generate the impregnation and/or coating. However, it is preferable to perform only 1 to 20 spray cycles, particularly preferably only 1 to 10 spray cycles. A reduced number of spray cycles means less consumption of material and permits faster and, in particular, less expensive production of the prostheses.

After the spraying, the vascular prosthesis may be dried, in particular by heat. For example, the vascular prosthesis can be dried at a temperature of 15 to 75° C., in particular 15 to 50° C., preferably 15 to 30° C.

The liquid provided for spraying the vascular prosthesis preferably has, in addition to the at least one sealing material, an organic solvent, which can be a ketone, in particular acetone and/or 3-pentanone, THF, chloroform or a mixture thereof.

To generate the impregnation and/or coating, the outer surface of the vascular prosthesis may be sprayed with a liquid that contains the at least one sealing material in a proportion of 1 to 50% by weight, in particular 5 to 25% by weight, preferably 7 to 15% by weight, relative to the total weight of the liquid.

The liquid may have a dynamic viscosity of 20 to 60 mPas, in particular 30 to 45 mPas, preferably 32 to 41 mPas. The aforesaid dynamic viscosity values are preferably measured on the basis of a solution of the at least one sealing material in an acetone solution at 25° C. Preferably, the solution has a concentration (weight/volume; w/v) of the at least one sealing material of 5 to 15% by weight, in particular 8 to 12% by weight, preferably 9 to 11% by weight, relative to the total volume of the solution.

The at least one sealing material is expediently a biocompatible material, in particular one that seals the prosthesis with respect to body fluids, in particular blood.

Preferably, both an impregnation in the prosthesis wall and also a coating on the outer surface of the prosthesis are generated within the scope of our methods.

The at least one sealing material that generates the impregnation and the at least one sealing material that generates the coating can be different. However, it is preferable if the same at least one sealing material is used to generate the impregnation and the coating.

The at least one sealing material is preferably resorbable. A sealing material of this kind has, on the one hand, the advantage that the amount of foreign material introduced is reduced again in the mid to long term after the implantation. On the other hand, the inflammatory processes triggered by the resorption process support the secondary anchoring of the vascular prosthesis by contributing to improved encapsulation of the prosthesis by connective tissue.

Moreover, the at least one sealing material can be a film-forming polymer, in particular a biopolymer.

The at least one sealing material can be of biological origin, in particular of animal origin, preferably of bovine, porcine and/or equine origin. Preferably, the at least one sealing material is chosen from the group consisting of collagen, gelatin, albumin and combinations thereof.

Preferably, however, the at least one sealing material is of synthetic origin. The at least one sealing material is preferably a synthetic polymer. Examples of suitable polymers are, in particular, synthetic polymers such as polyhydroxy alkanoates and copolymers thereof.

The term “copolymer” is to be understood as a polymer composed of two or more different types of monomer units. In line with this definition, the expression “copolymer” can therefore include a bipolymer, tripolymer, tetrapolymer or the like.

The at least one sealing material may be chosen from the group consisting of polyglycolide, polylactide, poly-ε-caprolactone, polytrimethylene carbonate, poly-para-dioxanone, poly-3-hydroxybutyrate, poly-4-hydroxybutyrate, copolymers thereof, stereoisomers, in particular diastereomers, thereof, salts thereof, and combinations, in particular blends, thereof.

The at least one sealing material may be a three-armed polyester having terminal hydroxyl groups from hydroxy acids that are polymerized onto a central trifunctional hydroxy compound, the three arms being tetrapolymers of lactide, ε-caprolactone, trimethylene carbonate and glycolide.

The lactide is preferably L-lactide.

The polymer is preferably composed of 30 to 45 mol % lactide, 20 to 40 mol % ε-caprolactone, 10 to 28 mol % trimethylene carbonate, and 3 to 25 mol %, in particular 10 to 25 mol %, glycolide.

Preferably, the polymer is a segmented polymer, in particular of three first segments connected to the trifunctional hydroxy compound and of three second segments connected to the free ends of the first segments, wherein the first segments are different from the second segments.

The first segment is preferably free of lactide. The second segment is preferably free of trimethylene carbonate.

The first segment is particularly preferably composed of ε-caprolactone, trimethylene carbonate and glycolide.

The second segment is preferably composed of lactide, glycolide and, optionally, ε-caprolactone.

The second segment is also preferably free of ε-caprolactone.

The first segment can contain 30 to 40 mol %, in particular 32 to 35 mol %, ε-caprolactone, relative to the total amount of the monomers in both segments.

Moreover, the first segment can contain 10 to 20 mol %, in particular 13 to 17 mol %, trimethylene carbonate, relative to the total amount of the monomers in both segments.

Furthermore, the first segment can contain 7 to 12 mol %, in particular 8 to 11 mol %, glycolide, relative to the total amount of the monomers in both segments.

The second segment preferably contains 30 to 45 mol %, in particular 32 to 42 mol %, lactide, relative to the total amount of the monomers in both segments.

The second segment can also be characterized in that it contains 0 to 4 mol %, in particular 1 to 4 mol %, ε-caprolactone, relative to the total amount of the monomers in both segments.

Moreover, the second segment can contain 1 to 10 mol %, in particular 2 to 8 mol %, glycolide, relative to the total amount of the monomers in both segments.

Further features and advantages of the three-armed polymer described above include those disclosed in WO 2008/058660 A2, the subject matter of which is incorporated by reference herein.

To improve or increase the kink stability, provision can also be made that the vascular prosthesis undergoes pleating. In principle, the vascular prosthesis can be pleated before and/or after being sprayed with the liquid.

Particularly advantageously, however, the vascular prosthesis undergoes pleating only once, specifically before being sprayed with the liquid. This is because we discovered that further pleating is no longer needed after the spraying process.

Further features and advantages of the method are set forth in the description below.

We also provide a vascular prosthesis produced or producible by our method. To avoid unnecessary repetition, reference is therefore made to the statements made above in connection with the method. For the rest, reference is made to the statements made below.

We further provide a vascular prosthesis with an inner surface and outer surface, a wall, and a sealing impregnation comprising at least one sealing material in the prosthesis wall and/or a sealing coating comprising at least one sealing material on the outer surface of the prosthesis.

When subjected to a force of 1 N acting perpendicularly with respect to the outer surface of the prosthesis, the prosthesis is preferably deformable such that the external diameter of the prosthesis decreases, in the direction of the acting force, by 60 to 100%, in particular 50 to 100%, preferably 70 to 100%, more preferably 80 to 100%, in relation to the original external diameter.

The expression “original external diameter” is to be understood as the external diameter that the vascular prosthesis has in the unloaded state, i.e. without a force acting on it, preferably from the outside.

The impregnation may be formed in the wall of the vascular prosthesis to a depth of 1 to 100%, in particular 50 to 100%, preferably 80 to 100%, relative to the wall thickness of the vascular prosthesis.

The impregnation can particularly advantageously perform the function of an adhesion bed or substrate (grounding or priming coat) for a coating optionally present on the outer surface of the vascular prosthesis.

The inner surface of the vascular prosthesis is preferably free of the at least one sealing material, in particular free of a continuous layer of the at least one sealing material, i.e. a layer partially or completely covering the inner surface of the prosthesis.

The impregnation and/or coating, in particular the coating, is preferably structured and not smooth, in particular not film-like.

The impregnation and/or coating, in particular the coating, particularly preferably has fibrous structures. The fibrous structures can have a diameter of 0.1 to 10 μm, in particular 0.4 to 5 μm, preferably 1.5 to 3 μm.

It is also preferable if the vascular prosthesis comprises the at least one sealing material in a proportion of 10 to 60% by weight, in particular 20 to 50% by weight, preferably 25 to 40% by weight, relative to the total weight of the vascular prosthesis.

If the vascular prosthesis only has an impregnation within the above meaning, the vascular prosthesis can then comprise the at least one sealing material in a proportion of 1 to 25% by weight, in particular 5 to 20% by weight, preferably 10 to 15% by weight, relative to the total weight of the vascular prosthesis.

In contrast, if the vascular prosthesis only has a coating within the above meaning, the vascular prosthesis can then comprise the at least one sealing material in a proportion of 9 to 35% by weight, in particular 15 to 30% by weight, preferably 15 to 25% by weight, relative to the total weight of the vascular prosthesis.

Advantageously, the vascular prosthesis has a water permeability of 0 to 20 ml/cm² min, in particular of 0 to 10 ml/cm² min, preferably 0 to 5 ml/cm² min.

The coating may have a thickness of 5 to 750 μm, in particular 10 to 300 μm, preferably 15 to 100 μm.

The impregnation and/or the coating may consist of the at least one sealing material.

However, it can be advantageous if the vascular prosthesis, in particular the impregnation and/or coating, has additives, in particular pharmaceutical compositions or medicaments, biologically active compounds, nanoparticles or the like.

Preferred additives can be chosen from the group consisting of cellular growth factors, cellular differentiation factors, cellular adhesion factors, cellular recruitment factors, antimicrobial substances, in particular antimicrobial metals such as silver, disinfecting substances, anti-inflammatory substances, antithrombogenic substances or anticoagulants, X-ray contrast media and combinations thereof.

The vascular prosthesis is preferably produced or formed as a textile vascular prosthesis, in particular a woven or knitted vascular prosthesis.

The wall of the vascular prosthesis may be formed from a material, in particular polymer, which is preferably chosen from the group consisting of polyester, polyamide, polyethylene, polypropylene, polyvinylidene difluoride, polyhexa-fluoropropylene, polytetrafluoropropylene, polytetra-fluoroethylene, in particular expanded polytetra-fluoroethylene (ePTFE), copolymers thereof, and combinations, in particular blends, thereof.

Preferred polyesters are chosen from the group consisting of polyethylene terephthalate (PET), polypropylene terephthalate (PPT), polybutylene terephthalate (PBT) and combinations, in particular blends, thereof. Polyethylene terephthalate (PET) is particularly preferred on account of its good biocompatibility and its excellent long-term stability.

Preferred materials for the wall of a textile vascular prosthesis are non-resorbable polyesters, in particular polyethylene terephthalate.

The vascular prosthesis may have an internal diameter of 2 to 50 mm, in particular 4 to 40 mm.

Further features and advantages will become clear from the following description of preferred examples, figures, the associated figure descriptions, and the appended claims. The features can in each case be implemented singly or in combination with each other.

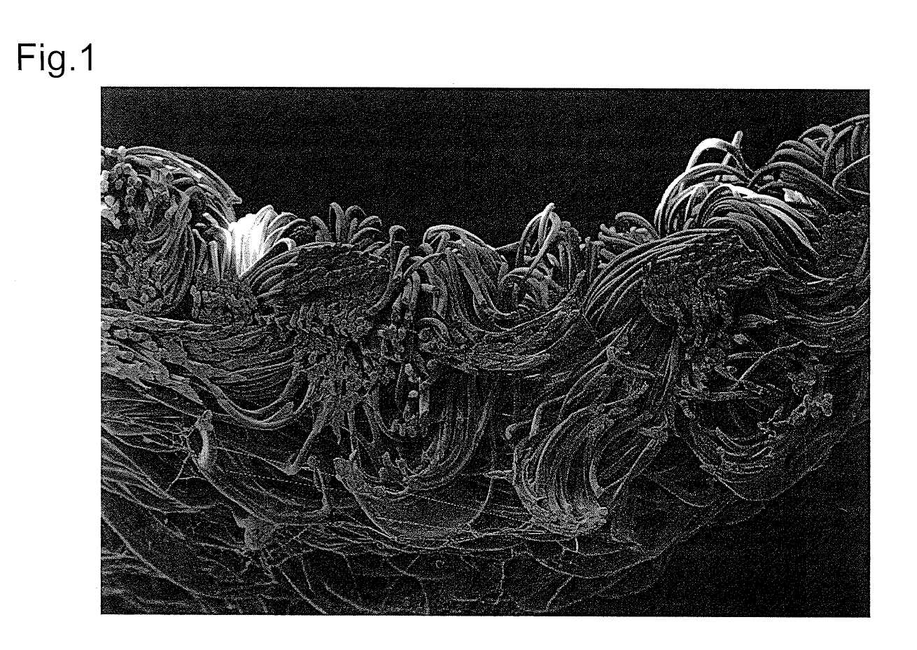

Turning now to the Drawings, FIG. 1 shows the scanning electron microscope image of a PET vascular prosthesis with an impregnation of a tetrapolymer, composed of 40 mol % L-lactide, 30 mol % ε-caprolactone, 26 mol % trimethylene carbonate and 4 mol % glycolide, in the prosthesis wall. To generate the polymer impregnation, the outer surface of the prosthesis was sprayed with an acetone solution containing the tetrapolymer (12.6% w/w) at a pressure of 0.1 bar, wherein one spray cycle was sufficient to generate a sealing impregnation. The prosthesis comprised the polymer in a proportion of ca. 11.5% by weight, relative to the total weight of the prosthesis.

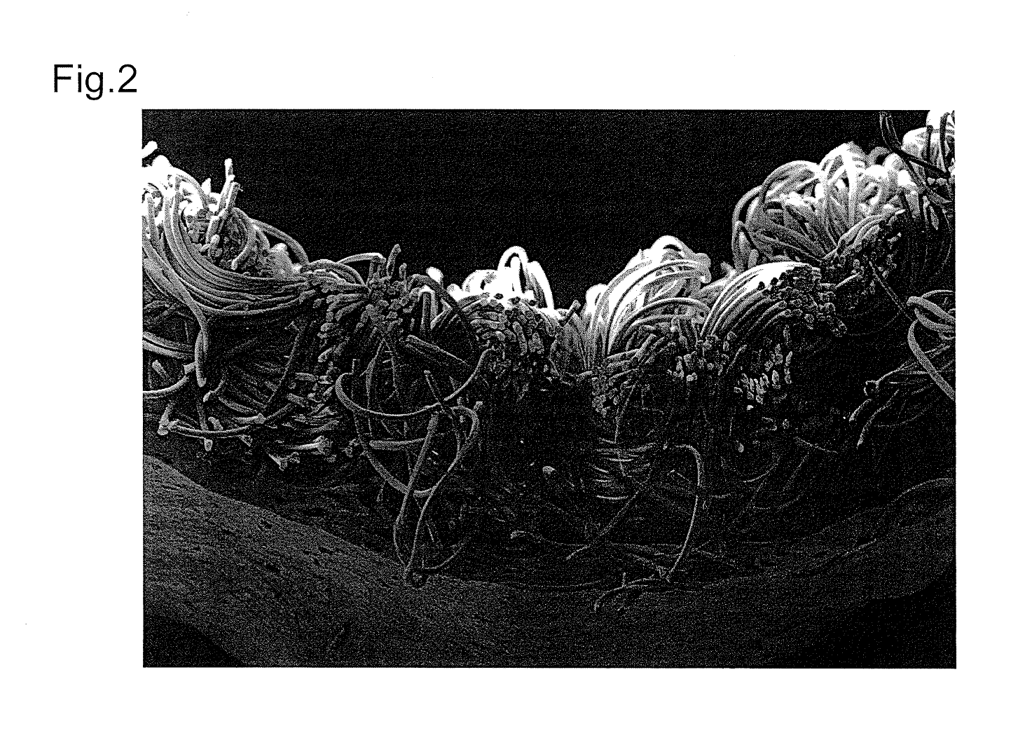

FIG. 2 shows the scanning electron microscope image of a PET vascular prosthesis, of which the outer surface is coated with a tetrapolymer composed of 40 mol % L-lactide, 30 mol % ε-caprolactone, 26 mol % trimethylene carbonate and 4 mol % glycolide. To generate the polymer coating, the vascular prosthesis was sprayed with an acetone solution containing the tetrapolymer (12.6% w/w) at a pressure of 7.5 bar. A total of 6 spray cycles were performed. The prosthesis comprised the coating polymer in a proportion of ca. 31.3% by weight, relative to the total weight of the prosthesis.

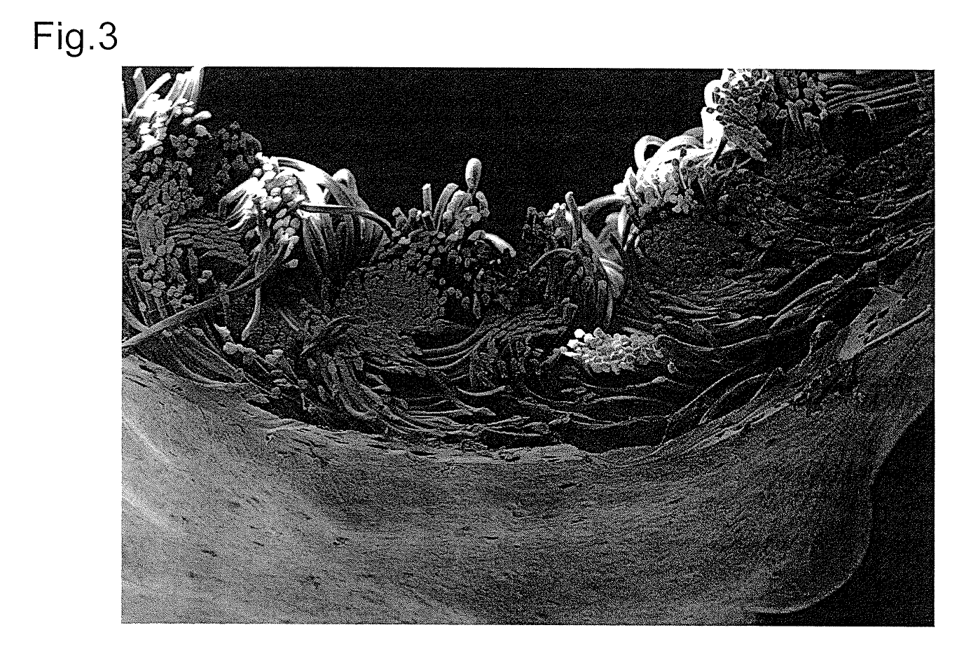

FIG. 3 shows the scanning electron microscope image of a PET prosthesis with an impregnation in the prosthesis wall and a coating on the outer surface of the prosthesis. The impregnation and the coating are formed from the same polymer, namely a tetrapolymer composed of 40 mol % L-lactide, 30 mol % ε-caprolactone, 26 mol % trimethylene carbonate and 4 mol % glycolide.

To form the impregnation, the vascular prosthesis was sprayed, in one spray cycle, with an acetone solution containing the tetrapolymer (12.6% w/w) at a pressure of 0.1 bar. To generate the coating on the outer surface, the prosthesis was sprayed, in 6 cycles and at a pressure of 7.5 bar, with an acetone solution containing the polymer (12.6% w/w).

Overall, the prosthesis comprised the polymer in a proportion of ca. 29.9% by weight.

### EXAMPLES

### 1. Softness Measurement of Vascular Prostheses

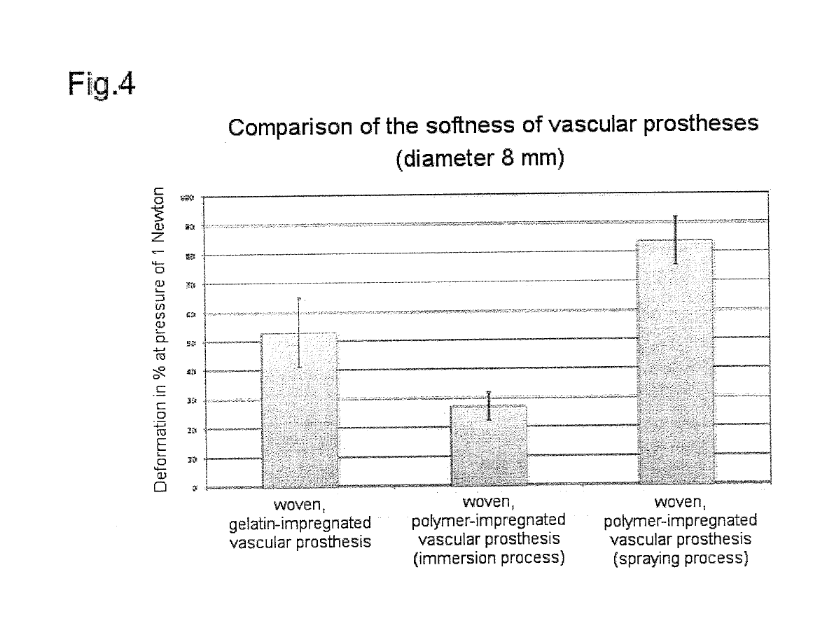

The results of the softness measurements are shown in graph form in FIG. 4. The ordinate indicates the deformation of the external diameters of the tested prostheses in % under the effect of a force of 1 Newton.

The softness measurements were carried out using a woven double-velour polyester vascular prosthesis (polyethylene terephthalate, PET; left-hand bar in FIG. 4) coated with gelatin in a conventional immersion process, a woven double-velour polyester prosthesis (PET; middle bar in FIG. 4) coated with a tetrapolymer of 40 mol % L-lactide, 30 mol % ε-caprolactone, 26 mol % trimethylene carbonate and 4 mol % glycolide in a conventional immersion process, and a double-velour polyester prosthesis (PET; right-hand bar in FIG. 4) coated with a tetrapolymer of 40 mol % L-lactide, 30 mol % ε-caprolactone, 26 mol % trimethylene carbonate and 4 mol % glycolide in a method according to the invention.

To coat the prostheses with the immersion process, the prostheses were immersed in an acetone solution containing the coating polymer, having a proportion of the coating polymer of 12.6% by weight, relative to the total weight of the solution.

To produce the prosthesis coated, an acetone solution containing the coating polymer, with a proportion of the coating polymer of 12.6% by weight, relative to the total weight of the solution, was sprayed onto the outer surface of an uncoated polyester prosthesis at a spray pressure of ca. 7.5 bar.

All of the vascular prostheses had an external diameter of ca. 8 mm.

To measure the softness, the vascular prostheses were fixed horizontally on a sample plate. A sample hammer having a bottom surface area of 10 mm×50 mm was then moved vertically downwards at a speed of 10 mm/min in the direction of the prostheses and pressed at a defined force of 1 N onto the prostheses. The deformation of the external diameter of the prostheses was measured in mm.

The measurement results obtained are shown in graph form in FIG. 4.

The measurement results shown in graph form in FIG. 4 illustrate that the external diameter of the coated prosthesis deformed to a significantly greater extent, under the effect of the force of 1 N, than in the conventionally coated vascular prostheses. In other words, the coated prosthesis is significantly softer than the conventionally coated prostheses.

Table 1 below contains some of the parameters used to characterize some of the prostheses used in the preceding series of tests:

TABLE 1
Characterization of some vascular prostheses

| | Prosthesis: (Coating method) | |
|---|---|---|
| | | Double-velour prosthesis |
| | Double-velour prosthesis | (High-pressure spraying |
| | (Immersion process) | method) |
| Prosthesis diameter: | 8 mm | 8 mm |
| Coating content: | 33.2% | 34.9% |
| Blood leaktightness: | present | present |

Table 1 shows that the vascular prosthesis also has the blood leaktightness required for vascular prostheses.

1-21. (canceled)

22. A method of producing a vascular prosthesis comprising spraying a vascular prosthesis having an inner surface, an outer surface and a wall with a liquid containing at least one sealing material, wherein the liquid is sprayed onto the outer surface at a pressure of 0.01 to 1.5 bar to generate a sealing impregnation in the wall, and/or is sprayed onto the outer surface at a pressure of 5.0 to 50 bar to generate a sealing coating on the outer surface.

23. The method according to claim 22, wherein the liquid is sprayed onto the outer surface at a pressure of 0.1 to 0.6 bar.

24. The method according to claim 22, wherein the liquid is sprayed onto the outer surface at a pressure of 6 to 9 bar.

25. The method according to claim 22, wherein the outer surface is sprayed with a liquid that contains the at least one sealing material in a proportion of 1 to 50% by weight relative to the total weight of the liquid.

26. The method according to claim 22, wherein, to generate the impregnation, the same at least one sealing material is used to generate the coating.

27. The method according to claim 22, wherein the at least one sealing material is resorbable.

28. The method according to claim 22, wherein the at least one sealing material is a film-forming polymer.

29. The method according to claim 22, wherein the at least one sealing material is of animal origin and selected from the group consisting of collagen, gelatin, albumin and combinations thereof.

30. The method according to claim 22, wherein the at least one sealing material is of synthetic origin and selected from the group consisting of polyglycolide, polylactide, poly-ε-caprolactone, polytrimethylene carbonate, poly-para-dioxanone, poly-3-hydroxybutyrate, poly-4-hydroxybutyrate, copolymers thereof and combinations thereof.

31. The method according to claim 22, wherein the at least one sealing material is a three-armed polyester having terminal hydroxyl groups from hydroxy acids polymerized onto a central trifunctional hydroxy compound, the three arms being tetrapolymers of lactide, e-caprolactone, trimethylene carbonate, and glycolide.

32. The method according to claim 22, further comprising pleating the prosthesis before the liquid is applied.

33. A vascular prosthesis, produced by the method according to claim 22.

34. A vascular prosthesis with an impregnation comprising at least one sealing material in a wall of the prosthesis and/or with a coating comprising at least one sealing material on an outer surface of the prosthesis, wherein the prosthesis, when subjected to a force of 1 N acting perpendicularly with respect to the outer surface, is deformed such that an external diameter of the prosthesis decreases in the direction of the acting force by 60 to 100% in relation to an original external diameter.

35. The vascular prosthesis according to claim 34, wherein the impregnation is formed in the wall to a depth of 1 to 100% relative to wall thickness of the prosthesis.

36. The vascular prosthesis according to claim 34, wherein the impregnation in the wall acts as an adhesion bed or substrate for the coating on the outer surface.

37. The vascular prosthesis according to claim 34, wherein the coating on the outer surface is structured and not smooth.

38. The vascular prosthesis according to claim 34, wherein the coating on the outer surface has fibrous structures.

39. The vascular prosthesis according to claim 38, wherein the fibrous structures have a diameter of 0.1 to 10 μm.

40. The vascular prosthesis according to claim 34, wherein the vascular prosthesis comprises the at least one sealing material in a proportion of 10 to 60% by weight relative to the total weight of the vascular prosthesis.

41. The vascular prosthesis according to claim 34, wherein the prosthesis has a water permeability of 0 to 20 ml/cm² min.

42. The vascular prosthesis according to claim 34, wherein the coating on the outer surface has a thickness of 5 to 750 μm.
